(19) United States
(12) Patent Application Publication
(10) Pub. No.: US 2025/0195829 A1
(43) Pub. Date: Jun. 19, 2025

(54) CATHETER COVER FOR URINARY CATHETER

(71) Applicant: ORLANDO HEALTH, INC., Orlando, FL (US)

(72) Inventors: Miclat Minerva, Orlando, FL (US); Jack Stubbs, Orlando, FL (US); Matthew Scott Solar, Maitland, FL (US)

(73) Assignee: ORLANDO HEALTH, INC., Orlando, FL (US)

(21) Appl. No.: 19/072,521

(22) Filed: Mar. 6, 2025

Related U.S. Application Data
(63) Continuation of application No. PCT/US2023/032268, filed on Sep. 8, 2023.
Application No. PCT/US2023/032268 is a continuation-in-part of application No. 17/940,255, filed on Sep. 8, 2022.

(51) Int. Cl. A61M 25/00

(52) U.S. Cl. CPC A61M 25/0017

(57) ABSTRACT

A cover for a urinary catheter may include at least one cover sheath, the at least one cover sheath including a longitudinal slit extending from a first end of the at least one cover sheath to a second end of the at least one cover sheath. The catheter cover may also include and a spring mechanically coupled to the at least one cover sheath and configured to retain the slit in a closed state, where the at least one cover sheath substantially surrounds an outer diameter surface of a urinary catheter when the cover sheath is in the closed state.

### CROSS-REFERENCE TO RELATED APPLICATIONS

This application is a continuation of PCT/US2023/032268, filed on Sep. 8, 2023, titled “CATHETER COVER FOR URINARY CATHETER”, published as WO 2024/054614 A4, which claims priority to U.S. application Ser. No. 17/940,255, filed Sep. 8, 2022, the entirety of both applications are hereby incorporated by reference herein.

### BACKGROUND

Urinary catheters of the type shown in FIG. 1 have been used successfully for many years. However, such catheters do have certain shortcomings. For example, common Foley catheters are indwelling catheters that remain in the body for days at a time, if not longer. These catheters need cleaning regularly (e.g., twice a day) via manual wiping via manual wiping with chlorhexidine wipes once a day and as needed. While such cleaning substantially reduces the chances of infection, it can be difficult and uncomfortable to clean areas very near to the body (e.g., near the patient's urethra). Further, since the catheter may slide slightly relative to the patient, the portions of the catheter immediately adjacent to the patient and/or immediately inside the patient are often overlooked and/or improperly cleaned. The embodiments discussed herein address these issues.

### BRIEF DESCRIPTION OF THE DRAWINGS

The embodiments discussed herein may be better understood with reference to the following drawings and description. The components in the figures are not necessarily to scale. Moreover, in the figures, like-referenced numerals designate corresponding parts throughout the different views.

FIG. 1 is an illustration showing a prior art urinary catheter.

FIG. 2 is an illustration showing a catheter cover for a urinary catheter or other catheter in accordance with certain aspects of the present disclosure, where a distal portion of the device is shown with a portion removed such that the internal area of the catheter cover is visible.

FIG. 3 is an illustration showing a second end of the catheter cover from FIG. 2.

FIG. 4 is an illustration showing a second end of the catheter cover from FIG. 2 having a portion removed such that the internal portion of the catheter cover is visible

FIG. 5 is an illustration showing a female body engagement portion of the catheter cover from FIG. 2

FIG. 6 is an illustration showing an additional view of the body engagement portion of the catheter cover from FIG. 2

FIG. 7 is an illustration of a catheter cover having a male body engagement portion in accordance with certain aspects of the present disclosure

FIG. 8 is an illustration of a catheter cover lacking a body engagement portion, but adapted for being secured to a selected body engagement portion, in accordance with certain aspects of the present disclosure.

FIG. 9 is an illustration of a catheter cover having another embodiment of a body engagement portion in accordance with certain aspects of the present disclosure.

FIGS. 10, 11, and 12 are illustrations showing three embodiments of catheter covers for surrounding a urinary catheter with three respective sealing features in accordance with certain aspects of the present disclosure.

FIGS. 13, 14, and 15 are illustrations showing three additional embodiments of catheter covers with multiple cover sheath layers for surrounding a urinary catheter with three respective sealing features in accordance with certain aspects of the present disclosure.

FIG. 16 is an illustration of another embodiment of the catheter cover with multiple cover sheath layers for surrounding a urinary catheter in accordance with certain aspects of the present disclosure.

### DETAILED DESCRIPTION

Various aspects are described below with reference to the drawings in which like elements generally are identified by like numerals. The relationship and functioning of the various elements of the aspects may be better understood by reference to the following detailed description. However, aspects are not limited to those illustrated in the drawings or explicitly described below. It also should be understood that the drawings are not necessarily to scale, and in certain instances, details may have been omitted that are not necessary for an understanding of aspects disclosed herein, such as conventional fabrication and assembly.

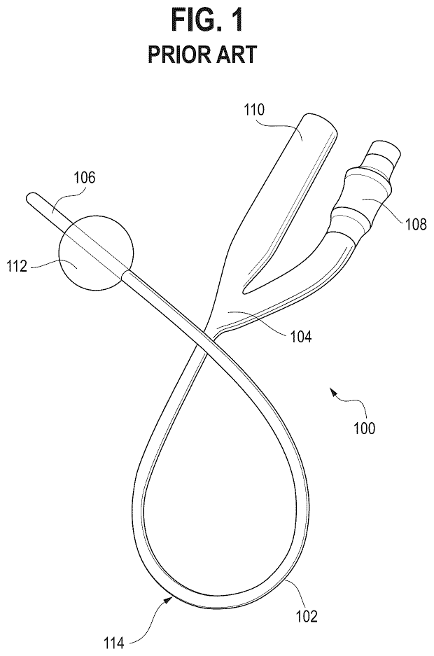

FIG. 1 is an illustration showing a urinary catheter 100 (e.g., a Foley catheter) common in the prior art. As shown, the urinary catheter 100 generally includes a catheter body 102 with a first end 104 and a second end 106. At the first end 104, the urinary catheter 100 includes an inflation port 108 and a drainage port 110. The drainage port 110 provides access to a central lumen of the urinary catheter 100 and may be the opening by which urine is drained from the patient. The drainage port 110 may also operate as an interface for receiving a drainage tube, which may lead to a collection chamber. The inflation port 108 is typically in fluid communication with an inflation balloon 112 via a cavity between a double-walled portion of the catheter body 102. When inflated, the balloon 112 secures the catheter in position within a patient's bladder.

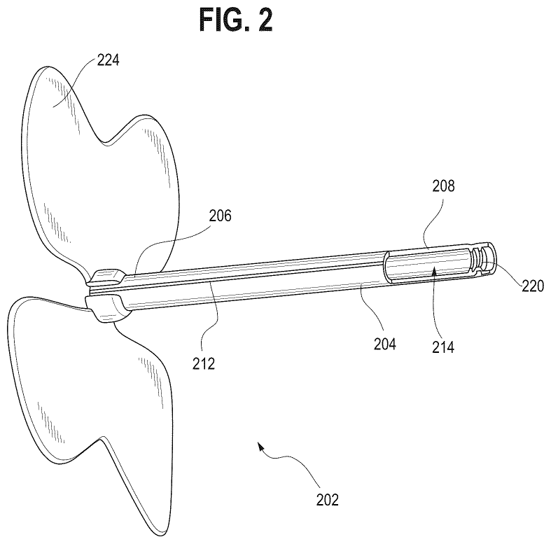

As discussed in the background above, urinary catheters of the type shown in FIG. 1 have been used successfully for many years but do have shortcomings. FIGS. 2-16 show various embodiments of catheter covers 202 that address these issues and provide an improved catheter environment. Notably, while the catheter covers 202 of this description primarily related to urinary catheters, similar catheter covers may be used for other types of catheters.

In particular, the catheter covers 202 may cover the portion of the urinary catheter 100 that is immediately adjacent to the patient to prevent soiling. Also, the catheter covers 202 may be removable (and perhaps disposable) such that they can be easily replaced, which may complement or potentially replace certain cleaning procedures. Further, the catheter covers 202 may engage the patient's body in a way such that sliding of the urinary catheter 100 relative to the patient is prevented or minimized, thereby enhancing the overall cleanliness of the urinary catheter 100.

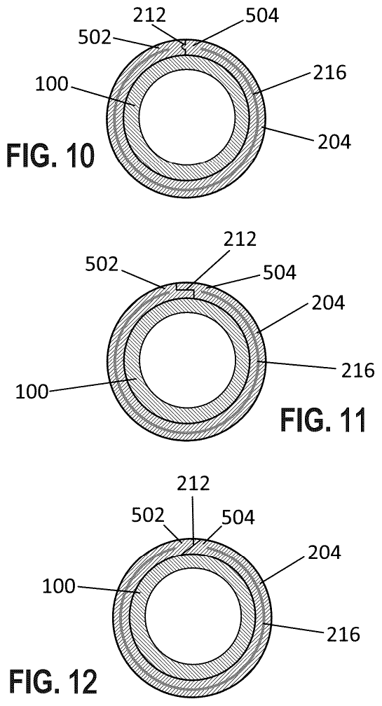

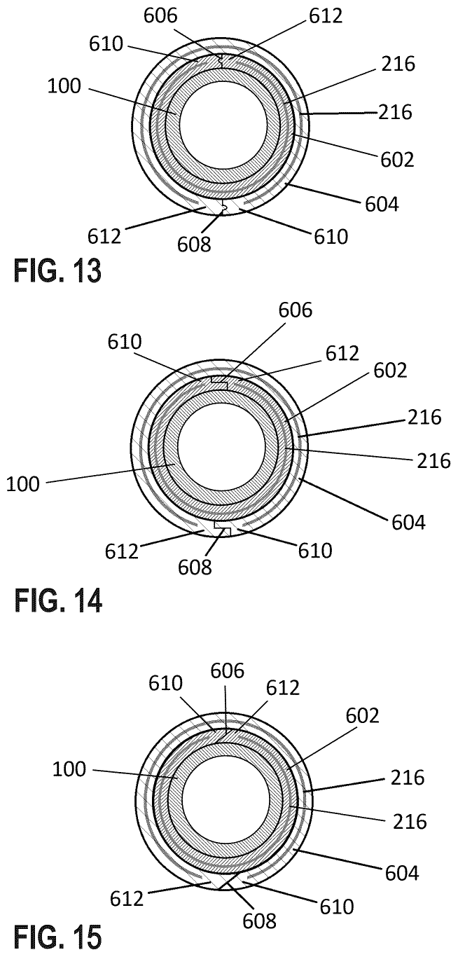

Referring to FIGS. 2-4, the catheter cover 202 generally includes an elongated cover sheath 204 having a first end 206 and a second end 208. The cover sheath 204 may be made of any suitable material (e.g., silicone, polymer-based synthetics, etc.). In some embodiments, the cover sheath 204 may include, or be coated with, a microbial agent and/or a microbial-limiting material (such as silver, copper, etc.). Also, it is contemplated that at least a portion of the cover sheath 204 may be transparent or translucent such that fluids within the urinary catheter remain visible. In certain embodiments disclosed herein, the cover sheath 204 may comprise a single-layer cover sheath, as is illustrated in FIGS. 10-12. Alternatively, the cover sheath may include multiple sheath layers such as two, three, four, five, or more sheath layers. In particular embodiments, as illustrated in more detail in FIGS. 13-16, the cover sheath 204 includes two cover sheath layers, wherein a first cover sheath 602 is wrapped around the urinary catheter 100, and wherein a second cover sheath 604 is wrapped around the entirety or a portion of the first cover sheath 602, thereby fully or partially enclosing both the first cover sheath 602 and the urinary catheter 100 therein along its length. In various embodiments, the outer diameter surface of the first cover sheath 602 may be attached or physically coupled to the inner diameter surface of the second cover sheath 604. The two layers may be adhered together, for example, in a permeant manner using a silicone based adhesive, e.g., a heat-curable silicone based adhesive. Other possibilities include a double-sided adhesive tape or other adhesives.

The first end 206 of the catheter cover 202 is intended to be located adjacent to the patient's body, typically at the entrance to the patient's urethra. Optionally, the terminus of the first end 206 may contact the patient's body such that no (or little) space is located between the patient's body and the device's first end 206. As discussed in more detail below, the catheter cover 202 may include a body engagement portion 224 at its second end 208 configured for enhanced cleanliness. The word “configured” as used herein identifies structural limitations including the dimensions and proportions of the configured item being made to align with and/or conform to another item, expressly including when such item is not part of the configured item and is not part of a claimed device (e.g., the catheter cover being configured to completely surround—or at least to substantially surround—an outer diameter surface of a urinary catheter means that the cover is of appropriate size and materials to fit all the way around or nearly all the way around a urinary catheter . . . where the cap can have absolute dimensions that differ in different embodiments to fit different catheters, but when claimed herein does not require presence of a catheter).

The catheter cover 202 may include a slit 212 along the length of the cover sheath 204. The slit 212 may be an elongated opening or other discontinuity that extends radially and generally interrupts the perimeter of the cover sheath 204. Advantageously, the slit 212 may provide the ability for the catheter cover 202 to be opened (e.g., via direct force from a medical professional's hands) and then wrapped around the outer diameter surface 114 of the urinary catheter 100 (e.g., once in place, the inner diameter surface 214 of the cover sheath 204 contacts the outer diameter surface of the urinary catheter 100).

In multiple-layer (e.g., two-layer) cover sheath embodiments, as illustrated in FIGS. 13-15, the first cover sheath 602 may include a slit 606, and the second cover sheath 604 (or other outermost cover sheath layer) may also include a slit 608 or an opening 614. As stated above, the first cover sheath 602 may be opened at the slit 606 and wrapped around the urinary catheter 100, and the second cover sheath 604 may be opened at its slit 608 or opening 614 and wrapped entirely or partially around the first cover sheath 602 after the first cover sheath 602 has been wrapped around the urinary catheter 100.

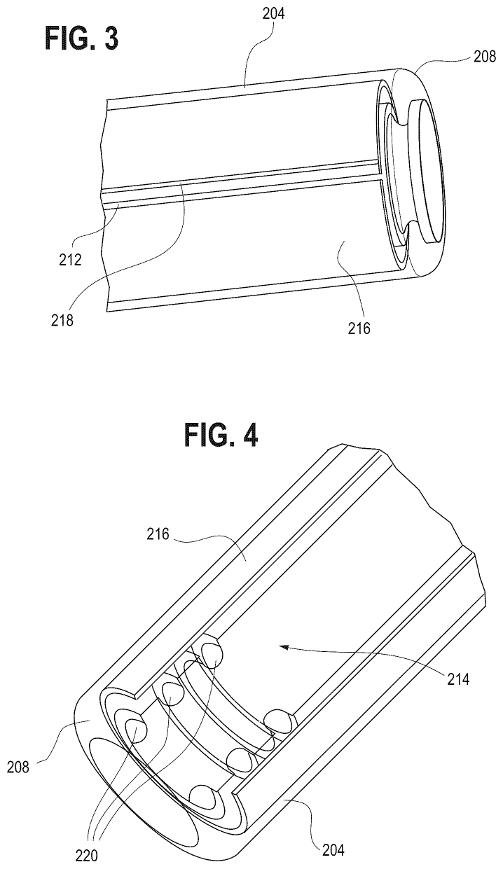

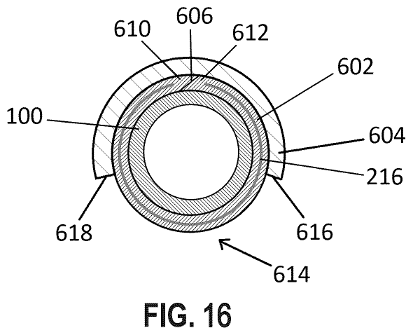

Referring to FIGS. 3-4, the catheter cover 202 may include a spring 216 that provides the cover sheath 204 with a tendency to wrap relatively tightly around the urinary catheter 100 when the catheter cover 202 is in its installed position. In multiple-layer (e.g., two-layer) cover sheath embodiments, as illustrated in FIGS. 13-15, the first cover sheath 602 and the second cover sheath 604 may both include the spring 216, though this is not necessarily required. For example, in multiple-layer embodiments, it is possible that only one of the layers (e.g., the first cover sheath 602 or the second cover sheath 604) includes the spring 216, while other cover sheath layers do not include the spring 216. FIG. 16 shows such an embodiment, wherein the first cover sheath 602 includes the spring 216, while the second cover sheath does not. Advantageously, the spring 216 may provide adequate engagement (via contact pressure) between the catheter cover 202 and the underlying urinary catheter 100 or underlying cover sheath layers such that slippage is prevented. In the depicted embodiment, the spring 216 is an elongated tube formed of a resilient material (e.g., a low-alloy steel or other metal, Nitinol, a resilient polyethylene or other synthetic material, etc.) and having a spring slit 218. Optionally, and as shown in FIGS. 3-4, the spring 216 may be inside, or surrounded by, the material forming the exposed surfaces (outer and inner) of the cover sheath 204, which may be advantageous for ensuring appropriate surface characteristics are not negatively affected by the spring (e.g., it may be desirable to cover the inner portion of the spring material with silicon for enhanced friction with the urinary catheter 100 and the outer portion of the spring to prevent the spring's metal from poking or otherwise contacting the patient).

Other spring types are also contemplated. For example, as an alternative (or in addition to) the spring 216, the catheter cover 202 may include a compression band (e.g., a rubber band) extending around the outer surface of the cover sheath 204, one or more magnets located at the slit 212, a tie extending around the circumference, or any other suitable device that imparts a tendency for the slit 212 or slits 606 and 608 in a multi-layer embodiment to close. In certain embodiments, a separate spring may not be necessary, but rather the material aspects (including resiliency) of the cover sheath 204 itself may be sufficient for closing the device and providing adequate engagement with the urinary catheter 100.

Optionally, the catheter cover 202 may include one or more friction-enhancing devices for preventing a sliding motion of the cover sheath 204 relative to the urinary catheter 100. For example, referring to FIG. 3-4, three protrusions 220 are located at the second end 208 of the catheter cover 202 and extend from the inner diameter surface 214 of the cover sheath 204. Thus, when installed, these protrusions 220 may directly contact the outer diameter surface 114 of the urinary catheter 100, in some cases compressing against the urinary catheter 100 and/or compressing the outer-surface material of the urinary catheter 100 (thereby further enhancing the static friction between the devices). The spring 216 may radially overlap at least one of these protrusions 220 to enhance the contact pressure between the protrusions 220 and the urinary catheter 100, as shown.

While the protrusions 220 are located at the second end 208 of the device, they may be located in any other suitable location (i.e., anywhere along the length of the cover sheath's inner surface). For example, it is contemplated that substantially the entirety of the inner surface of the cover sheath 204 may include such protrusions for even more static friction. Also, it should be appreciated that more or fewer than three protrusions 220 may be included. The protrusions 220 may be unitary/integral with the cover sheath 204 such that they include the same material as that of the exposed surfaces of the cover sheath 204, but this is not required. By contrast, in other embodiments, similar protrusions may be separately assembled to the cover sheath 204 (e.g., O-rings may be placed in grooves within the inner-facing surface of the cover sheath 204). Similarly, a protrusion assembly including the protrusions may be separately formed and adhered to the cover sheath 204, e.g. to an end of the cover sheath 204 via an adhesive or welding process.

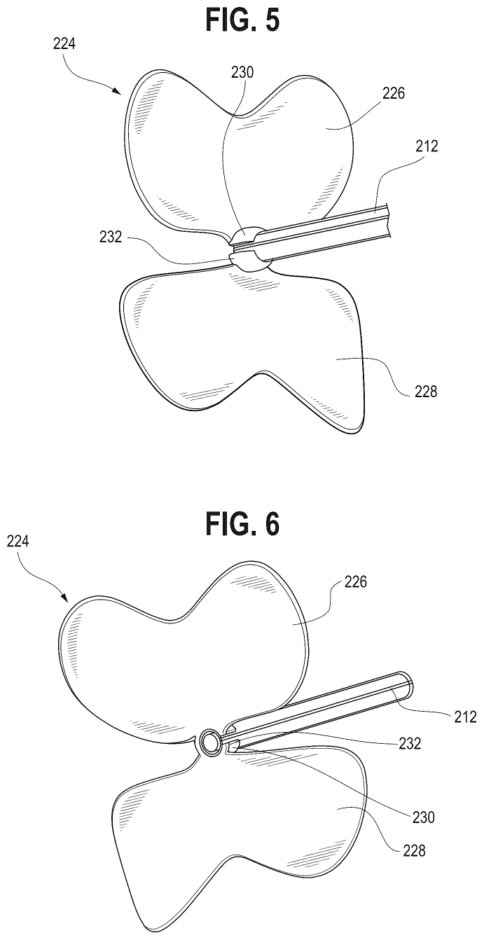

Optionally, the catheter cover 202 may include a body engagement portion, such as those shown in FIGS. 5-9. As discussed in more detail below, the body engagement portion (if included) may generally press against the patient and be adapted for the patient's particular anatomy. Advantageously, this device may prevent soiling of the urinary catheter near the urethral area and may substantially prevent or limit sliding action of the urinary catheter relative to the patient's urethra due to sufficient contact pressure with the patient in combination with high static friction relative to the urinary catheter.

Referring to FIGS. 5-6, the particular depicted body engagement portion includes a female contact device 224 generally sized and shaped for adapting to the anatomy surrounding a human female's urethral area. In particular, the device may include a set of panels, such as a first side 226 and a second side 228, which generally provides an organic profile and shape to fit to the anatomy of a female so that it is comfortable and covering.

One unique feature of the body engagement portion is the collar 230 where the body engagement portion is secured to the cover sheath 204. In a multiple-layer (e.g., two-layer) cover sheath embodiment, the collar 230 may be secured to the outermost cover sheath layer (e.g., the second cover sheath 604). As shown, the collar 230 may substantially surround the cover sheath 204, and an inward radial force imparted by the collar 230 on the cover sheath 204 may generally prevent the body engagement portion from moving relative to the cover sheath 204. In a multiple-layer (e.g., two-layer) cover sheath embodiment, the collar 230 may substantially surround the outermost cover sheath layer (e.g., the second cover sheath 604), and the inward radial force may be imparted by the collar 230 on the outermost cover sheath layer (e.g., the second cover sheath 604) may generally prevent the body engagement portion from moving relative to that outermost cover sheath layer. Optionally, the collar 230 may include a spring (much like the springs 216 discussed above), but this may not be required depending on the resilience and strength of the material used to form the collar 230. Further, the collar 230 may include a collar slit 232, which may radially align with the slit 212 of the cover sheath 204. In a multiple-layer (e.g., two-layer) cover sheath embodiment, the collar slit 232 may radially align with the slit (e.g., slit 608) or opening (e.g., opening 614) of the outermost cover sheath layer (e.g., the second cover sheath 604).

Advantageously (or additionally), the collar 230 may be glued or otherwise adhered to the cover sheath 204. In a multiple-layer (e.g., two-layer) cover sheath embodiment, the collar 230 may be glued or otherwise adhered to the outermost cover sheath layer (e.g., the second cover sheath 604).

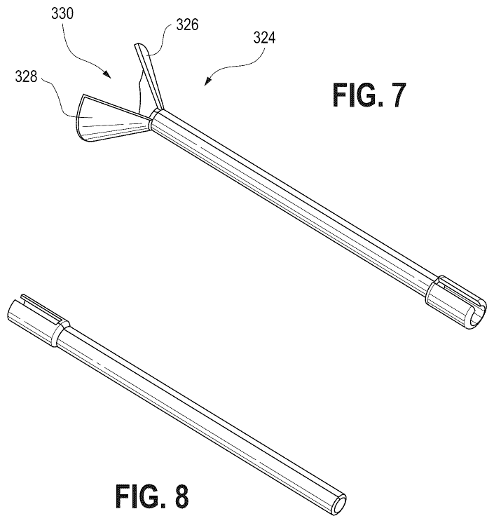

FIG. 7 shows another embodiment of a body engagement portion, which in this instance is a male contact device 324 configured for use on a male patient. As shown, the male contact device 324 includes a funnel-like structure having a first side 326 and a second side 328 that, when in use, generally cover the tip of the patient's penis. Advantageously, the funnel-like structure includes a slit or opening 330 that allows the device to open during installation around the urinary catheter 100. In various embodiments, as with the female contact device 224 discussed above, the slit or opening 330 may radially align with the slit 212 of the cover sheath 204. In a multiple-layer (e.g., two-layer) cover sheath embodiment, the slit or opening 330 may radially align with the slit (e.g., slit 608) or opening (e.g., opening 614) of the outermost cover sheath layer (e.g., the second cover sheath 604). The male contact device 324 may be removeable such that the same catheter cover may be connectable to an appropriate body engagement portion (e.g., male, female, those of different sizes, or the like). E.g., a catheter cover lacking a body engagement portion, but adapted for being secured to a selected body engagement portion, is shown in FIG. 8. As discussed throughout, this catheter cover may comprise a single-layer cover sheath arrangement, or a multiple-layer (e.g., two-layer) cover sheath arrangement.

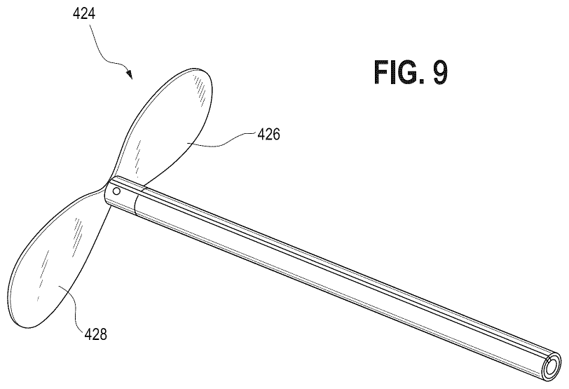

FIG. 9 shows another embodiment of a catheter cover having another embodiment of a body engagement portion 424. In this example, the body engagement portion 424 includes two wings or extensions 426 and 428 that extend radially away from a collar 430, which may be advantageous for manipulating body tissue of the female anatomy. Notably, when different body engagement portions are contemplated, these body engagement portions may be formed separately and may be attachable to the cover sheath (e.g., via the discussed collar). In other words, the body engagement portions may remain modular. This may be advantageous for allowing a medical professional to select an appropriate body-engagement portion that is appropriately sized and shaped for the patent while using a standardized cover sheath, which could be a single-layer cover sheath arrangement, or a multiple-layer (e.g., two-layer) cover sheath arrangement in various embodiments.

FIGS. 10-12 show three single-layer embodiments of the catheter cover 202 surrounding the urinary catheter 100, particularly to illustrate three of many possible ways to create a seal at the slit 212 of the cover sheath 204. For example, referring to FIG. 10, a first side 502 of the cover sheath 204 includes a depression that corresponds to a protrusion of a second side 504 of the cover sheath 204. In FIG. 11, the first side 502 of the cover sheath 204 includes a first portion that extends beneath a corresponding second portion of the second side 504. For example, in various embodiments, the second portion may overlap the first portion along an extended length of the circumference of the cover sheath 204 to provide increased sealing and a longer pathway to help avoid ingress of debris and bacteria. For example, the second portion may overlap the first portion along a range between 5° and 180° around the perimeter or circumference of the cover sheath 204, with specific examples including 5°, 10°, 15°, 30°, 45°, 60°, 75°, 90°, 105°, 120°, 135°, 150°, 165°, and 180°. In FIG. 12, complementary angled surfaces of the first side 502 and the second side 504 engage one another. In a similar manner as discussed with regard to FIG. 11, the angled surfaces may be at such an obtuse or gradual angle (and possibly curved to follow the curve of the cover sheath 204) so that the first side 502 may overlap the second side 504 along a range between 5° and 180° around the perimeter or circumference of the cover sheath 204, with specific examples including 5°, 10°, 15°, 30°, 45°, 60°, 75°, 90°, 105°, 120°, 135°, 150°, 165°, and 180°. In each of these embodiments, the slit 212 is shaped such that there is no linear, radial path towards the urinary catheter 100 available for unwanted debris. Many other embodiments with different slit shapes are also contemplated.

In order to provide additional sealing against ingress of unwanted debris or bacteria toward the urinary catheter 100, multiple-layer cover sheath arrangements are disclosed in accordance with certain embodiments. FIGS. 13-16 show four example multiple-layer cover sheath arrangement embodiments of the catheter cover 202 surrounding the urinary catheter 100. Specifically, FIGS. 13-16 illustrate two-layer cover sheath arrangements, but it is contemplated that one or more additional cover sheath layer may also be included. As discussed above, the cover sheath 204 may include multiple layers of cover sheaths including, for example, a first cover sheath 602 and a second cover sheath 604. The first cover sheath 602 may be wrapped around the urinary catheter 100 such that it substantially surrounds the outer diameter surface 114 of the urinary catheter 100 when the first cover sheath 602 is in the closed state. The first cover sheath 602 may have an inner diameter that is substantially the same as or slightly larger (e.g., within a tolerance range) than the outer diameter surface 114 of the urinary catheter 100. Once in place, the inner diameter surface of the first cover sheath 602 contacts the outer diameter surface 114 of the urinary catheter 100.

As is shown in FIGS. 13-15, the second cover sheath 604 may be wrapped around the first cover sheath 602 such that it substantially surrounds the outer diameter surface of the first cover sheath 602 when the second cover sheath 604 is in the closed state. Alternatively, as is shown in FIG. 16, the second cover sheath 604 may be wrapped at least partially around the first cover sheath 602 such that it at least partially surrounds the outer diameter surface of the first cover sheath 602.

Advantageously, as is shown in each of FIGS. 13-16, the second cover sheath 604 may be positioned on the first cover sheath 602 such that an uninterrupted portion of the second cover sheath 604 (e.g., a portion that does not include the slit 608 or opening 614) covers the slit 606 in the first cover sheath 602. In this manner, the second cover sheath 604 may help keep the slit 606 in the first cover sheath 602 closed, and may provide a second barrier or extended pathway to avoid ingress of debris and bacteria to the urinary catheter 100.

In various approaches, the inner diameter of the inner surface of second cover sheath 604 is substantially the same as or slightly larger (e.g., within a tolerance range) than the outer diameter of the outer surface of the first cover sheath 602. In this manner, a seal is effectively formed between these two adjacent and contacting surfaces to further prevent the ingress of debris or bacteria that may happen to pass the sealed slit 608 of the second cover sheath 604, thereby helping to prevent such debris or bacteria from even contacting the slit 606 of the first cover sheath 602. This seal may be improved by adhering the inner surface of second cover sheath 604 to the outer surface of the first cover sheath 602 along a circumferential portion of the interfacing surfaces on one side of the slit 606 in the first cover sheath 602. For example, a permanent adhesive such as a silicone based adhesive can be used. During installation, a second adhesive may be applied between the interfacing surfaces (e.g., between the inner surface of the second cover sheath 604 and the outer surface of the first cover sheath 602) on the second, opposite side of the slit 606 in the first cover sheath 602. This second adhesive may be applied, for example, by applying a liquid adhesive, or by removing a backing layer from a layer of double-sided tape on one of the inner surface of the second cover sheath 604 or the outer surface of the first cover sheath 602.

The embodiments of FIGS. 13-15 illustrate three different possible arrangements to seal the slits in each of the first cover sheath 602 and the second cover sheath 604, which correspond to the three embodiments discussed above with respect to FIGS. 10-12. In particular, FIG. 13 illustrates a first example sealing arrangement. The first cover sheath 602 includes a first slit 606, and the second cover sheath 604 includes a second slit 608. The first slit 606 and the second slit 608 may both be sealed in accordance with the sealing mechanism as disclosed with respect to FIG. 10, wherein first sides 610 of the cover sheaths include a depression that corresponds to a protrusion of second sides 612 of the cover sheaths.

FIG. 14 illustrates a second example sealing arrangement. The first slit 602 and the second slit 604 may both be sealed in accordance with the sealing mechanism as disclosed with respect to FIG. 11, wherein first sides 610 of the cover sheaths include a portion that extends beneath a corresponding portion of the second sides 612 of the cover sheaths.

FIG. 15 illustrates a third example sealing arrangement. The first slit 606 and the second slit 608 may both be sealed in accordance with the sealing mechanism as disclosed with respect to FIG. 12, wherein complementary angled surfaces of the first sides 610 and the second sides 612 of the cover sheaths engage one another.

In certain embodiments, as illustrated in FIGS. 13-15, the slit 606 of the first cover sheath 602 is arranged circumferentially offset from the slit 608 of the second cover sheath 604. In particular, the two slits 606 and 608 may be arranged on approximate opposite sides of the urinary catheter 100. Stated another way, the two slits 606 and 608 may be arranged 180° from each other around the perimeter or circumference of the urinary catheter 100. However, other arrangements are possible.

Further, while not expressly illustrated in the drawings, different combinations of seals are contemplated amongst the different cover sheath layers. For example, the first cover sheath 602 may include the depression and protrusion arrangement illustrated in FIGS. 13 and 10, while the second cover sheath 604 may include the sealing arrangement of FIGS. 14 and 11, or the sealing arrangement of FIGS. 15 and 12. Similarly, the first cover sheath 602 may include the sealing arrangement illustrated in FIGS. 14 and 11, while the second cover sheath 604 may include the sealing arrangement of FIGS. 13 and 10, or the sealing arrangement of FIGS. 15 and 12. Similarly still, the first cover sheath 602 may include the sealing arrangement illustrated in FIGS. 15 and 12, while the second cover sheath 604 may include the sealing arrangement of FIGS. 13 and 10, or the sealing arrangement of FIGS. 14 and 11. Different combinations are contemplated and are considered to be within the scope of the present disclosure.

FIG. 16 illustrates another embodiment wherein the second cover sheath 604 does not substantially surround the entirety of the outer diameter surface of the first cover sheath 602, but instead only partially surrounds the outer diameter surface of the first cover sheath 602. In this approach, an opening 614 or gap may be formed between the first end 616 and second end 618 of the second cover sheath 604. For example, the second cover sheath 605 may surround and overlap the first cover sheath 602 along a range between 5° and 350° around the perimeter or circumference of the first cover sheath 602, with specific examples including 5°, 10°, 15°, 30°, 45°, 60°, 75°, 90°, 105°, 120°, 135°, 150°, 165°, 180°, 185°, 190°, 195°, 210°, 225°, 240°, 255°, 270°, 285°, 300°, 315°, 330°, 345°, and 350°. In some examples, the overlap is at least 180° around the perimeter or circumference of the first cover sheath 602.

As explained above, the second cover sheath 604 may be positioned or situated such that an uninterrupted portion of the second cover sheath 604 (e.g., a portion that does not include the opening 614) covers the slit 606 in the first cover sheath 602. In certain embodiments, as illustrated in FIG. 16, the center of the opening 614 and the slit 606 in the first cover sheath 602 may be arranged 180° from each other around the perimeter or circumference of the urinary catheter 100. Alternatively, the orientation of the opening 614 may be offset relative to the slit 606 such that the second cover sheath 604 overlaps the first cover sheath 602 more on one side of the slit 606 than the other side of the slit 606.

In various embodiments, the first cover sheath 602 and second cover sheath 604 are separate sheaths, which may be molded or extruded. In an alternative embodiment, the first cover sheath 602 and second cover sheath 604 are formed from a single molded or extruded item such that the first cover sheath 602 and second cover sheath 604 are joined together in a single formed assembly. For example, a portion of the inner surface of the second cover sheath 604 may be integrally formed with a corresponding portion of the outer surface of the first cover sheath 602. This integrally formed portion should be formed only on one side of the slit 606 in the first cover sheath 602 in order to allow access to the slit 606 so that the urinary catheter 100 can be placed within the first cover sheath 602 through the slit 606.

While not shown, it is also contemplated that an optional locking device may be included at the slit 212 to prevent the cover sheath 204 from opening (although the spring 212 or other features discussed above may be sufficient on its own). For example, it is contemplated that a light adhesive may be applied to the seam. Other embodiments may include tape, a zipper, a hook, threads, wraps, or any other suitable locking device.

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

One general aspect includes a catheter cover for a urinary catheter, at least one cover sheath, the at least one cover sheath including a longitudinal slit extending from a first end of the at least one cover sheath to a second end of the at least one cover sheath. The catheter cover may also include a spring mechanically coupled to the at least one cover sheath and configured to retain the slit in a closed state, where the at least one cover sheath is configured to substantially surround an outer diameter surface of a urinary catheter when the at least one cover sheath is in the closed state.

Implementations may include one or more of the following features individually or in combination with one another. The spring may include an elongated tube having a spring slit that is coextensive with the slit of the at least one cover sheath. The spring may substantially surround by a material forming the at least one cover sheath. The catheter cover may include at least one friction protrusion located at the second end of the at least one cover sheath, the at least one friction protrusion configured to contact the outer diameter surface of a urinary catheter when the at least one cover sheath is in the closed state. The spring may radially overlap the at least one friction protrusion. The at least one friction protrusion may be integral with an inner surface of the at least one cover sheath. The catheter cover may also include a body engagement portion extending radially outward from the at least one cover sheath and located at the first end of the catheter cover. The body engagement portion may include a collar securing the body engagement portion to the c at least one over sheath. The collar may include a collar slit that is coextensive with the slit of the at least one cover sheath. The body engagement portion may be modular relative to the at least one cover sheath. The longitudinal slit of the at least one cover sheath may be configured to close such that a first side of the longitudinal slit contacts a second side of the longitudinal slit when in a closed state surrounding a urinary catheter.

Another general aspect may include a catheter cover for a urinary catheter, at least one cover sheath, the at least one cover sheath including a longitudinal slit extending from a first end of the at least one cover sheath to a second end of the at least one cover sheath. The catheter cover also may include and a body engagement portion extending radially outward from the at least one cover sheath and located at the first end of the catheter cover.

Implementations may include one or more of the following features individually or in combination with one another. The catheter cover may include a spring mechanically coupled to the at least one cover sheath, where the spring may include an elongated tube having a spring slit that is coextensive with the slit of the at least one cover sheath. The catheter cover may include at least one friction protrusion located at the second end of the at least one cover sheath, the at least one friction protrusion configured to contact an outer diameter surface of a urinary catheter when the at least one cover sheath is in a closed state. The body engagement portion may include a collar securing the body engagement portion to the at least one cover sheath, and where the collar may include a collar slit that is coextensive with the slit of the at least one cover sheath. The body engagement portion may be modular relative to the at least one cover sheath. The body engagement portion may be adapted for an anatomy of a female patient and may include a set of panels. The body engagement portion may be adapted for an anatomy of a male patient and may include a funnel-like structure. The longitudinal slit of the at least one cover sheath may be configured to close such that a first side of the longitudinal slit contacts a second side of the longitudinal slit when in a closed state surrounding a urinary catheter.

Implementations of any of the aspects discussed above may also include one or more of the following features individually or in combination with one another. The at least one cover sheath may comprise at least a first cover sheath and a second cover sheath, wherein the first cover sheath includes the longitudinal slit extending from the first end to the second end, and wherein the second cover sheath includes a longitudinal slit or an opening extending from the first end to the second end, wherein the first cover sheath is configured to substantially surround the outer diameter surface of a urinary catheter, and wherein the second cover sheath at least partially surrounds an outer diameter surface of the first cover sheath and covers at least the slit in the first cover sheath. The longitudinal slit of the first cover sheath and the longitudinal slit or opening of the second cover sheath may be oriented on opposite circumferential sides of a urinary catheter from each other. The first cover sheath and the second cover sheath may both include the spring. The longitudinal slit of the first cover sheath may be configured to close such that a first side of the longitudinal slit contacts a second side of the longitudinal slit when in a closed state surrounding a urinary catheter, and the longitudinal slit of the second cover sheath may be configured to close such that a first side of the longitudinal slit contacts a second side of the longitudinal slit when in a closed state surrounding the first cover sheath.

Another general aspect may include a catheter cover including a first cover sheath including a longitudinal slit extending from a first end of the first cover sheath to a second end of the first cover sheath, and a second cover sheath including a longitudinal slit extending from a first end of the second cover sheath to a second end of the second cover sheath. The first cover sheath may be configured to substantially surround an outer diameter surface of a catheter, and the second cover sheath may be configured to substantially surround an outer diameter surface of the first cover sheath.

Implementations may include one or more of the following features individually or in combination with one another. The longitudinal slit of the first cover sheath and the longitudinal slit of the second cover sheath may be oriented on opposite circumferential sides of a urinary catheter from each other. The first cover sheath and the second cover sheath may both include a spring mechanically coupled to each cover sheath and configured to retain each slit in a closed state. The longitudinal slit of the first cover sheath may be configured to close such that a first side of the longitudinal slit of the first cover sheath contacts a second side of the longitudinal slit of the first cover sheath when in a closed state surrounding a urinary catheter, and the longitudinal slit of the second cover sheath may be configured to close such that a first side of the longitudinal slit of the second cover sheath contacts a second side of the longitudinal slit of the second cover sheath when in a closed state surrounding the first cover sheath. The present claims are directed to a catheter cover, which is defined and claimed on its own with reference to—but not including—a catheter such as a urinary catheter, except where a catheter is claimed as part of a medical device system.

Another general aspect includes a method that includes forming a urinary catheter consistent with any of the aspects discussed above.

We claim:

1. A catheter cover for a urinary catheter, comprising:
at least one cover sheath, the at least one cover sheath including a longitudinal slit extending from a first end of the at least one cover sheath to a second end of the at least one cover sheath; and
a spring mechanically coupled to the at least one cover sheath and configured to retain the slit in a closed state, wherein the at least one cover sheath is configured to substantially surround an outer diameter surface of a urinary catheter when the at least one cover sheath is in the closed state.

2. The catheter cover of claim 1, wherein the spring includes an elongated tube having a spring slit that is coextensive with the slit of the at least one cover sheath.

3. The catheter cover of claim 2, wherein the spring is substantially surrounded by a material forming the at least one cover sheath.

4. The catheter cover of claim 1, further comprising at least one friction protrusion located at the second end of the at least one cover sheath, the at least one friction protrusion configured to contact the outer diameter surface of a urinary catheter when the at least one cover sheath is in the closed state.

5. The catheter cover of claim 4, wherein the spring radially overlaps the at least one friction protrusion.

6. The catheter cover of claim 4, wherein the at least one friction protrusion is integral with an inner surface of the at least one cover sheath.

7. The catheter cover of claim 1, further comprising a body engagement portion extending radially outward from the at least one cover sheath and located at the first end of the catheter cover.

8. The catheter cover of claim 7, wherein the body engagement portion includes a collar securing the body engagement portion to the at least one cover sheath.

9. The catheter cover of claim 8, wherein the collar includes a collar slit that is coextensive with the slit of the at least one cover sheath.

10. The catheter cover of claim 7, wherein the body engagement portion is modular relative to the at least one cover sheath.

11. The catheter cover of claim 1,
wherein the longitudinal slit of the at least one cover sheath is configured to close such that a first side of the longitudinal slit contacts a second side of the longitudinal slit when in a closed state surrounding a urinary catheter.

12. The catheter cover of claim 1,
wherein the at least one cover sheath comprises at least a first cover sheath and a second cover sheath,
wherein the first cover sheath includes the longitudinal slit extending from the first end to the second end, and wherein the second cover sheath includes a longitudinal slit or an opening extending from the first end to the second end,
wherein the first cover sheath is configured to substantially surround an outer diameter surface of a urinary catheter, and
wherein the second cover sheath at least partially surrounds an outer diameter surface of the first cover sheath and covers at least the slit in the first cover sheath.

13. The catheter cover of claim 12,
wherein the longitudinal slit of the first cover sheath and the longitudinal slit or opening of the second cover sheath are oriented on opposite circumferential sides of a urinary catheter from each other.

14. The catheter cover of claim 1,
wherein the first cover sheath and the second cover sheath both include the spring.

15. The catheter cover of claim 1,
wherein the longitudinal slit of the first cover sheath is configured to close such that a first side of the longitudinal slit of the first cover sheath contacts a second side of the longitudinal slit of the first cover sheath when in a closed state surrounding a urinary catheter, and
wherein the longitudinal slit of the second cover sheath is configured to close such that a first side of the longitudinal slit of the second cover sheath contacts a second side of the longitudinal slit of the second cover sheath when in a closed state surrounding the first cover sheath.

16. A catheter cover for a urinary catheter, comprising:
at least one cover sheath, the at least one cover sheath including a longitudinal slit extending from a first end of the at least one cover sheath to a second end of the at least one cover sheath; and
a body engagement portion extending radially outward from the at least one cover sheath and located at the first end of the catheter cover.

17. The catheter cover of claim 16, further comprising a spring mechanically coupled to the at least one cover sheath, wherein the spring includes an elongated tube having a spring slit that is coextensive with the slit of the at least one cover sheath.

18. The catheter cover of claim 16, further comprising at least one friction protrusion located at the second end of the at least one cover sheath, the at least one friction protrusion configured to contact an outer diameter surface of a urinary catheter when the at least one cover sheath is in a closed state.

19. The catheter cover of claim 16, wherein the body engagement portion includes a collar securing the body engagement portion to the at least one cover sheath, and wherein the collar includes a collar slit that is coextensive with the slit of the at least one cover sheath.

20. The catheter cover of claim 16, wherein the body engagement portion is modular relative to the at least one cover sheath.

21. The catheter cover of claim 16, wherein the body engagement portion is adapted for an anatomy of a female patient and includes a set of panels.

22. The catheter cover of claim 16, wherein the body engagement portion is adapted for an anatomy of a male patient and includes a funnel-like structure.

23. The catheter cover of claim 16,
wherein the longitudinal slit of the at least one cover sheath is configured to close such that a first side of the longitudinal slit contacts a second side of the longitudinal slit when in a closed state surrounding a urinary catheter.

24. The catheter cover of claim 16,
wherein the at least one cover sheath comprises at least a first cover sheath and a second cover sheath,
wherein the first cover sheath includes the longitudinal slit extending from the first end to the second end, and wherein the second cover sheath includes a longitudinal slit or an opening extending from the first end to the second end,
wherein the first cover sheath is configured to substantially surround an outer diameter surface of a urinary catheter, and
wherein the second cover sheath at least partially surrounds an outer diameter surface of the first cover sheath and covers at last the slit in the first cover sheath.

25. The catheter cover of claim 24,
wherein the longitudinal slit of the first cover sheath and the longitudinal slit or opening of the second cover sheath are oriented on opposite circumferential sides of a urinary catheter from each other.

26. The catheter cover of claim 16,
wherein the first cover sheath and the second cover sheath both include the spring.

27. The catheter cover of claim 16,
wherein the longitudinal slit of the first cover sheath is configured to close such that a first side of the longitudinal slit of the first cover sheath contacts a second side of the longitudinal slit of the first cover sheath when in a closed state surrounding a urinary catheter, and
wherein the longitudinal slit of the second cover sheath is configured to close such that a first side of the longitudinal slit of the second cover sheath contacts a second side of the longitudinal slit of the second cover sheath when in a closed state surrounding the first cover sheath.

28. A method, comprising:
forming a catheter cover for a urinary catheter,
wherein the catheter cover includes at least one cover sheath, the at least one cover sheath including a longitudinal slit extending from a first end of the at least one cover sheath to a second end of the at least one cover sheath, and
wherein the catheter cover includes a spring mechanically coupled to the at least one cover sheath and configured to retain the slit in a closed state, wherein the at least one cover sheath substantially surrounds an outer diameter surface of a urinary catheter when the at least one cover sheath is in the closed state.

29. The method of claim 28, wherein the spring includes an elongated tube having a spring slit that is coextensive with the slit of the at least one cover sheath.

30. The method of claim 28, wherein the spring includes a body engagement portion extending radially outward from the at least one cover sheath and located at the first end of the catheter cover.

31. A catheter cover comprising:
a first cover sheath including a longitudinal slit extending from a first end of the first cover sheath to a second end of the first cover sheath; and
a second cover sheath including a longitudinal slit or opening extending from a first end of the second cover sheath to a second end of the second cover sheath,
wherein the first cover sheath is configured to substantially surround an outer diameter surface of a catheter, and
wherein the second cover sheath is configured to at least partially surround an outer diameter surface of the first cover sheath and cover at least the slit in the first cover sheath.

32. The catheter cover of claim 31,
wherein the longitudinal slit of the first cover sheath and the longitudinal slit or opening of the second cover sheath are oriented on opposite circumferential sides of a catheter from each other.

33. The catheter cover of claim 31,
wherein the first cover sheath and the second cover sheath both include a spring mechanically coupled to each cover sheath and configured to retain the slit in the first cover sheath in a closed state.

34. The catheter cover of claim 31,
wherein the longitudinal slit of the first cover sheath is configured to close such that a first side of the longitudinal slit of the first cover sheath contacts a second side of the longitudinal slit of the first cover sheath when in a closed state surrounding a catheter, and
wherein the longitudinal slit of the second cover sheath is configured to close such that a first side of the longitudinal slit of the second cover sheath contacts a second side of the longitudinal slit of the second cover sheath when in a closed state surrounding the first cover sheath.

35. A medical device system comprising:
a catheter; and
a catheter cover according to claim 31.
